(19) United States
(12) Patent Application Publication
(10) Pub. No.: US 2006/0020027 A1
(43) Pub. Date: Jan. 26, 2006

(54) Use of curcumanoids as histone acetyltransferases (HATs) inhibitors

(76) Inventors: Karanam Balasubramanyam, Bangalore (IN); Radihika A. Varier, Bangalore (IN); Altaf M, Bangalore (IN); Swaminathan V, Bangalore (IN); Tapas Kumar Kundu, Bangalore (IN)

Correspondence Address: CHOATE, HALL & STEWART LLP, TWO INTERNATIONAL PLACE, BOSTON, MA, 02110, US

(21) Appl. No.: 11/149,694

(22) Filed: Jun. 10, 2005

(30) Foreign Application Priority Data
Jun. 11, 2004 (IN) 544/CHE/2004

(51) Int. Cl. A61K 31/255; A61K 31/225; A61K 31/15; A61K 31/12

(52) U.S. Cl. 514/517; 514/548; 514/678; 514/640

(57) ABSTRACT

Compounds having the formula 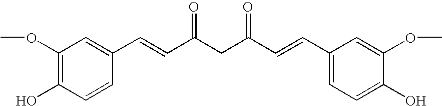 and derivatives thereof are used to inhibit histone acetyltransferases.

### FIELD OF INVENTION

This invention relates to the field of novel anticancer therapeutics, which can also be used for treating several other diseases (HIV, cardiac hypertrophy, asthma) in humans.

### PRIOR ART

The eukaryotic genome is packaged into a highly complex nucleoprotein structure, chromatin. Though apparently repressive, a precise organization of chromatin is essential for replication, repair, recombination and chromosomal segregation. Alteration in chromatin organization modulates the expression of underlying genes, which also includes the genes of integrated viral genomes (1). These dynamic changes in the chromatin structure are brought about by post-translational modifications of the amino terminal tails of the histones and the ATP-dependent chromatin remodeling (2). Specific amino acids within the histone tail are the sites of a variety of modifications including phosphorylation, acetylation and methylation. Among these, acetylation of histones and nonhistone proteins play a pivotal role in the regulation of gene expression. A balance between acetylation and deacetylation states of these proteins forms the basis of the regulation of transcription. Dysfunction of histone acetyltransferases and histone deacetylases is often associated with the manifestation of several diseases, which include cancer, cardiac hypertrophy and asthma (3-5). These enzymes, therefore, are potential new targets for therapy.

Histone acetyltransferases (HATs) modulate gene expression by catalyzing targeted acetylation of the ε-amino group of lysine residues on histones and nonhistone proteins. HATs can be classified into several families on the basis of number of highly conserved structural motifs. These include the GNAT family (Gcn5-related N-acetyltransferase e.g. Gcn5, PCAF), the MYST (MOZ, YBF2/SAS3 and TIP60) group and the p300/CBP family (4, 6). The p300 and CBP are ubiquitously expressed, global transcriptional coactivators that have critical roles in a wide variety of cellular phenomenon including cell cycle control, differentiation and apoptosis (7, 8). The transcriptional coactivator function of these two proteins is partially facilitated by their intrinsic HAT activity (9). Significantly, p300/CBP also acetylates several nonhistone proteins with functional consequences. The most notable example is the acetylation of p53. p300/CBP directly interacts with p53 and acetylates the tumor suppressor in vivo and in vitro to enhance its transcriptional activation ability (4, 10) and consequently DNA repair. Mutation in the HAT active site abolishes transactivation capability of p300/CBP (11). Analysis of colorectal, gastric and epithelial cancer samples show that in several instances there is a mis-sense mutation as well as deletion mutations in the p300 gene (12). 80% of the glioblastoma cases have been associated with the loss of heterozygosity of the p300 gene (13). In acute myeloid leukemia (AML), the gene for CBP is translocated and fused to either the Monocytic Leukemia Zinc finger (MOZ) gene or to MLL (A homeotic regulator, mixed lineage leukemia). In both cases, the HAT activity of CBP remains intact. However, the fusion proteins cause aberrant gene expression through improper targeting of the genes. Retention of partial HAT function by the fusion protein may result in the altered regulation of the target gene, while a loss of function of the fusion protein may result in the normal gene not being transcribed at all. In either case, this result s in aberrant cell cycle regulation leading to cancer (4). Mutations in HATs cause several other disorders apart from cancer. Rubinstein-Taybi syndrome (RTS) is found to be a resultant of mutations in CBP. A single mutation at the PHD-type zinc finger in the HAT domain of CBP, resultsing in an alteration of a conserved amino acid (E1278K), causes this syndrome. Interestingly, this mutation in CBP also abolishes its HAT activity (14, 15). The degradation of p300/CBP is also found to be associated with certain neurodegenerative diseases (16). Though the role of HAT activity in cardiac hypertrophy is still speculative, overexpression of p300 is sufficient to induce hypertrophy. The HAT domain of p300 was found to be essential for the stimulation of hypertrophy (17). The hyperacetylation of histones were also observed in the lung cells under asthmatic conditions (17). Chromatin analysis of the HIV genome shows that a single nucleosome (nuc-1) located at the transcription start site gets specifically disrupted during transcriptional activation. Treatment of the cell lines latently infected with HIV-1, with histone deacetylase inhibitors (e.g., trichostatin A/trapoxin/valproic acid) causes a global acetylation of the cellular histones. Consequently this treatment also results in the transcriptional activation of the HIV promoter and a robust increase in virus production (18, 19). Furthermore, recruitment of HDAC1 close to nuc-1 by host factors YY1 and LSF to the HIV-1 LTR have been shown to inhibit transcription by maintaining nuc-1 in the hypoacetylated state (20, 21). Taken together these data establish the fact that histone acetylation of nuc-1 is at least partly essential for the multiplication of HIV-1. Acetylation of the HIV-1 transactivator Tat by p300, PCAF and human GCN5 has also been demonstrated to be important for HIV transcriptional activity (22, 23, 24 and reviewed in 25). Therefore both HAT and HDAC modulators (activator/inhibitor) could serve as new generation anti-HIV therapeutics.

Significant progress has been made in the field of histone deacetylase inhibitors as antineoplastic drugs and also against cardiac hypertrophy. However, there are very few inhibitors of histone acetyltransferases known so far. Availability of recombinant HATs made it possible to synthesize and test more target-specific inhibitors, Lys-CoA for p300 and H3-CoA-20 for PCAF (26). Though it has been extensively employed for in vitro transcription studies, cells are not permeable to Lys-CoA (27). Recently, we have isolated the first naturally occurring HAT inhibitor, anacardic acid, from cashew nut shell liquid and garcinol from Garcinia indica, which are non-specific inhibitors of p300/CBP and PCAF but are capable of easily permeating the cells in culture (28, 29). Different chemical modifications of these inhibitors were attempted to identify enzyme-specific inhibitors but it serendipitously lead to the synthesis of the only known p300 specific activator, CTPB. Here we describe the discovery of curcumin as the first p300/CBP specific cell permeable HAT inhibitor. We have shown that it does not affect the HAT activity of PCAF as well as histone deacetylase and methyltransferase activities. However, p300 HAT activity dependent chromatin transcription was efficiently repressed by curcumin but not the transcription from DNA template. It could also inhibit the acetylation of histones in vivo. Significantly, curcumin repressed the multiplication of human immunodeficiency virus 1 (HIV1) and also inhibited the acetylation of HIV-Tat protein.

### SUMMARY OF INVENTION

Acetylation of histones and non-histone proteins is an important post-translational modification involved in the regulation of gene expression in eukaryotes and all viral DNA that integrates into the human genome, e.g. the human immunodeficiency virus (HIV). Dysfunction of histone acetyltransferases is often associated with the manifestation of several diseases. In this respect, HATs are the new potential targets for the design of therapeutics. Here we report that curcumin (diferuloylmethane), a major curcumanoid in the spice turmeric, is a specific inhibitor of the p300/CBP histone acetyltransferase (HAT) activity but not of PCAF, in vitro and in vivo. Furthermore, curcumin could also inhibit the p300-mediated acetylation of p53 in vivo. It specifically represses the p300/CBP HAT activity-dependent transcriptional activation from chromatin but not a DNA template. Significantly, it could inhibit the acetylation of HIV-Tat protein in vitro by p300 as well as proliferation of the virus, as revealed by the repression in syncytia formation upon curcumin treatment in SupT1 cells. Thus non-toxic curcumin, which targets p300/CBP, may serve as a lead compound in combinatorial HIV therapeutics.

### EXPERIMENTAL PROCEDURES

Isolation and Purification of Curcumin from Curcuma Longa:

Twenty-five grams of curcumina longa in 100 mL of dichloromethane was mechanically stirred and refluxed for one hour. The mixture was suction filtered and the filtrate was concentrated in rotary evaporator maintained at 50° C. The reddish yellow oily residue was trituted with 20 mL of hexane and the resulting solid was collected by suction filtration. The crude material obtained after tritution with hexane was dissolved in minimum amount of 99% dichloromethane-1% methanol (v/v) and loaded onto a column packed with 75 gm of silica gel. The column was eluted with the same solvent. The fractions containing least polar colored components were combined and solvents were removed on a water bath to give curcumin. The purity and identity of the compound was determined by mass spectroscopy and NMR spectroscopy. The purified compound was stored in room temperature and dissolved freshly in DMSO for each use.

(mp 178-182° C.), ¹H NMR (DMSO-d₆) δ 3.90 (6H, s, OCH₃), 6.06 (1H, s, C(OH)=CH), 6.76 (2H, d 2.6), 7.32 (2H, s), 2H, d, 17-H), 9.70 (2H, Phenolic OH)

Histone acetyltransferase Assay: HAT assays were performed as described previously (30). 2.4 μg of highly purified HeLa core histones were incubated in HAT assay buffer containing 50 mM Tris-HCl, pH 8.0, 10% (v/v) glycerol, 1 mM dithiothreitol, 1 mM phenylmethylsulphonyl fluoride (PMSF), 0.1 mM EDTA pH 8.0, 10 mM sodium butyrate at 30° C. for 10 min. with or without baculovirus expressed recombinant p300/CBP or PCAF in the presence and absence of curcumin followed by addition of 1 μl of 4.7 Ci/mmol [³H]-acetyl CoA and were further incubated for another 10 min. in a 30 μl reaction. The reaction mixture was then blotted onto P-81 (Whatman) filter paper and radioactive counts were recorded on a Wallac 1409 liquid scintillation counter. The radiolabeled acetylated histones were visualized by resolving on 15% SDS-polyacrylamide gel and subjected to fluorography followed by autoradiography.

Histone methyltransferase and deacetylase Assays: Histone methyltransferase assays were performed in a 30 μl reaction. The reaction mixtures containing 20 mM Tris, 4 mM EDTA, pH 8.0, 200 mM NaCl and 2 μl (˜8 μg of protein/μl) HeLa nuclear extract or 2 μg of bacterially expressed HMTase domain of G9a fused to GST (31) were incubated in the presence or absence of curcumin for 10 min at 30° C. After the initial incubation, 1 μl of 8.3 Ci/mmol ³H—S— Adenosyl Methionine was added to the reaction mixtures and the incubation continued for 1 hour (in the case of nuclear extract) or for 15 minutes (in the case of G9a). The reaction products were TCA precipitated, resolved on 15% SDS-PAGE and subjected to fluorography followed by autoradiography.

For the deacetylation assays histones were acetylated by the recombinant p300 (20 ng) using 2.4 μg of core histones and 1 μl of 4.7 Ci/mmol [³H]-acetyl CoA in HAT assay buffer without sodium butyrate for 30 min at 30° C. (29) The activity of p300 was inhibited by incubating the reaction mixture with 5 μM p300 specific inhibitor Lysyl-CoA (20) for 15 min at 30° C., after which 50 ng of baculovirus expressed recombinant HDAC1 was added in the presence or absence of curcumin and incubated further for 45 min at 30° C. The samples were processed as described above.

In Vitro Chromatin Assembly: Chromatin template for in vitro transcription experiments was assembled and characterized as described earlier (9).

In vitro Transcription Assay: Transcription assays were carried out as described elsewhere (9) with the necessary modifications. The schematic representation of the in vitro transcription protocol is given in FIG. 4A. The reconstituted chromatin template (containing 30 ng DNA) or an equimolar amount of histone—free DNA was incubated with 50 ng of activator (Gal4-VP16) in a buffer containing 4 mM HEPES (pH 7.8), 20 mM KCl, 2 mM DTT, 0.2 mM PMSF, 10 mM sodium butyrate, 0.1 mg/ml bovine serum albumin, 2% glycerol. Baculovirus expressed recombinant full length p300 was preincubated with indicated amounts of curcumin at 20° C. for 20 min following which it was added to the transcription reaction and the acetylation reaction was carried out for 30 min at 30° C. HeLa nuclear extract (5 μl, which contains 8 mg/ml protein) was added then to initiate the preinitiation complex formation. Transcription reaction was started by the addition of NTP-mix and α-[³²P] UTP after the preinitiation complex formation and incubated further for 40 min at 30° C. For loading control separate reaction was setup with ˜25 ng of supercoiled ML200 DNA, and the transcription assay was carried out as described above, without the addition of the activator (Gal4-VP16) and 2 μl of this reaction was added to each of the transcription reactions. Reactions were terminated by the addition of 250 μl of stop buffer (20 mM Tris-HCl pH 8.0, 1 mM EDTA, 100 mM NaCl, 1% SDS and 0.025 ng/μl tRNA). Transcripts were analysed by 5% Urea-PAGE and quantification of transcription was done by phosphoimager (Fuji) analysis. To visualize the transcripts the gels were exposed to X-ray films.

Acid/Urea/Triton (A UT) polyacrylamide gel electrophoresis and Western Blotting for in vivo acetylated histones: HeLa cells (3×10⁶ cells per 90 mm dish) were seeded overnight and histones were extracted from 24 hours of compound treated cells as reported earlier (29,32). Briefly, cells were harvested, washed in ice-cold buffer A (150 mM KCl, 20 mM HEPES, pH 7.9, 0.1 mM EDTA and 2.5 mM MgCl₂) and lysed in buffer A containing 250 mM sucrose and 1% (v/v) Triton X 100. Nuclei were recovered by centrifugation, washed, and proteins were extracted for 1 h using 0.25 M HCl. The proteins were precipitated with 25% (w/v) trichloroacetic acid (TCA) and sequentially washed with ice-cold acidified acetone (20 μl of 12N HCl in 100 ml acetone), and acetone, air-dried and dissolved in the sample buffer (5.8 M urea, 0.9 M glacial acetic acid, 16% glycerol, and 4.8% 2-mercaptoethanol). The histones (equal amounts in all lanes) were resolved on AUT gel as described elsewhere (33,34).

For western blotting, the quantitated protein samples were run on a 12% SDS-polyacrylamide gel and following electrophoresis, proteins on the gel were electrotransferred onto an immobilon membrane (PVDF; Millipore Corp., Bedford, Mass.). The membranes were then blocked in 5% skim milk powder solution in 1×PBS containing 0.05% Tween 20 and then immunoblotted with anti-acetyl H3 (Calbiochem), anti-acetyl H4 (a kind gift from Dr. Alaine Verrault) and anti-H3 respectively. Detection was performed using goat anti-rabbit secondary antibody (Bangalore Genei) and bands were visualized by ECL detection system (Pierce).

Apoptosis assay: Curcumin-induced apoptosis was monitored by the extent of nuclear fragmentation. Nuclear fragmentation was visualized by Hoechst staining of apoptotic nuclei. The apoptotic cells were collected by centrifugation, washed with PBS and fixed in 4% paraformaldehyde for 20 minutes at room tempertature. Subsequently the cells were washed and resuspended in 20 μl PBS before depositing it on poly lysine-coated coverslips and were left to adhere on cover slips for 30 min at room temperature after which the cover slips were washed twice with PBS. The adhered cells were then incubated with 0.1% Triton X-100 for 5 min at room temperature and rinsed with PBS for three times. The coverslips were treated with Hoechst 33258 for 30 minutes at 37° C., rinsed with PBS and mounted them on slides with glycerol-PBS and processed as described previously (29).

Transient Transfection and Immunoprecipitation:

293 T cells were transfected with CMV-p53 and CMV-p300 using Lipofectamine 2000 reagent (Invitrogen) according to the manufacturer's instructions. The medium was replaced and the cells were incubated for an additional 24 hours with curcumin (100 μM) or vehicle (DMSO). The cells were harvested using RIPA buffer (150 mM NaCl, 50 mM Tris pH 7.4, 1% NP40, 0.1% Sodium deoxycholate, 1 mM EDTA, 0.5 μg/ml leupeptin, 0.5 μg/ml aprotinin and 0.5 μg/ml pepstatin) and the p53 protein was immunoprecipitated from the lysates using mouse monoclonal anti-p53 antibody, DO1 (oncogene). The immunocomplexes were bound on protein G-sepharose beads (Amersham Pharmacia) for 12 hours at 4° C., washed 3 times with RIPA buffer and subjected to SDS-PAGE on a 10% gel followed by western blotting using mouse monoclonal anti-p53 antibody, pAb421 or mouse monoclonal anti-acetylated lysine antibody.

Syncytium inhibition assay: The cell lines SupT1 and H9/HTLV-IIIb NIH 1983 were obtained from The AIDS Research and Reference Reagent Program. H9/HTLV-IIIb NIH 1983 cells carrying a stably integrated provirus were cocultured with excess numbers of SupT1 cells at a ration of 1:200 or 1:400. A total of 1×10⁶ cells were seeded per well in a 24-well plate and cultured in RPMI medium supplemented with 10% fetal calf serum and antibiotics. Curcumin in DMSO was added to the cells to the final concentration as shown and the cultures were incubated at 37° C. All the wells received the same amount of DMSO including the control wells. Formation of syncytia was visible under light microscope within 12 hrs. The total number of syncytia in 10 representative wells was counted at different time points (12, 24 and 48 h) and data for 12 h time point are presented. Identical results were obtained at other time points. All the assays were performed in triplicate wells and the experiment was performed two times.

### RESULTS AND DISCUSSION

Screening of plant extracts known to possess anticancer properties lead us to a polyphenolic compound from Curcuma longa rhizome, which is a potent and specific inhibitor of histone acetyltransferases p300/CBP, but not of PCAF. By employing mass spectroscopy and NMR spectroscopy, it was identified as curcumin. As can be seen from both the filter binding (FIG. 1A) and gel (FIG. 1B, C and D) HAT assays, the acetylation of histones H3 and H4 by p300 was strongly inhibited by curcumin (FIG. 1A, B and C lanes 5-7), with an IC₅₀ of approximately 25 μM, whereas the PCAF HAT activity showed no change even in the presence of 100 μM curcumin (FIGS. 1A and D). This data establishes curcumin as the first known p300/CBP specific natural HAT inhibitor.

In order to ensure absolute enzyme specificity, we went on to check the effect of curcumin on HDAC1 and histone methyltransferase activities. Deacetylation of acetylated core histones by recombinant baculovirus expressed histone deacetylase 1(HDAC1) was not effected by the presence of curcumin (FIG. 2A, lane 3 versus lanes 7 and 8 and FIG. 2B, lane 2 versus lanes 4 and 5). We have also investigated the effect of curcumin on histone methyltransferase activity. HeLa core histones were methylated with ³H—S-adenosyl methionine (SAM) by recombinant lysine methyltransferase G9a that specifically methylates lysine residues 9 and 27 of histone H3 (31) in the presence or absence of curcumin. As depicted in FIG. 2B histone methylation by G9a remains same in presence or absence of curcumin (FIG. 2B, lane 3 versus lanes 4-6). Furthermore, to find out whether curcumin has any effect on other histone methyltransferases, we have performed HMTase assay using HeLa nuclear extract as a source of other histone methyltransferase enzymes. Similar to G9a-mediated methylation of core histones, methylation by NE showed no difference whatsoever in the presence or absence of curcumin (FIG. 2C, lane 3 versus lanes 4-6). Taken together these results suggest that curcumin is absolutely specific for the histone acetyltransferase activity (of p300/CBP) but not other enzymes for which histones are the substrate. It also indicates that curcumin presumably binds to the enzyme rather than the substrate, histones.

We went on to characterize the nature of inhibition of curcumin on the p300 HAT activity. Enzyme kinetics was studied to understand the mechanism of curcumin-mediated inhibition of p300 HAT activity by changing both acetyl CoA (FIG. 3A) and histone concentrations (FIG. 3B) keeping the other constant at a time. In both the cases Kₘ, Vₘₐₓ and K_{cat} decreased indicating that curcumin does not bind to the active sites of either histones or acetyl CoA, but to some other site on the enzyme. Presumably, this binding site on p300/CBP is specific for curcumin and binding leads to a conformational change, resulting in a decrease in the binding efficiency of both histones and acetyl CoA to p300. In this connection, curcumin behaves in a unique manner as compared to another polyphenolic cell-permeable HAT inhibitor, garcinol, in which case upon changing the concentration of acetyl CoA, the inhibitor acts as an uncompetitive type while for core histones, as a competitive inhibitor (29).

To investigate the effect of curcumin on transcription, in vitro transcription experiments were performed using DNA and reconstituted chromatin template. Transcription from the DNA template was not affected by curcumin even at 300 μM concentration (FIG. 4B, lane 3 versus lanes 4-7). Significantly, increasing concentrations of curcumin repressed p300 HAT activity dependent chromatin transcription upto 3 fold at 100 μM and upto 8 fold at 300 μM. (FIG. 4C, lane 4 versus lanes 6 and 9). Taken together these data suggest that curcumin is a potent and specific inhibitor of p300 HAT activity in the transcriptional context.

Curcumin permeates the cell and it is known to have a role in cancer chemoprevention and also in tumor growth suppression (35). Exposure of tumor cells to curcumin in vitro results in the inhibition of cell proliferation and also induction of apoptosis (36). Consistent with the previous reports on other cell lines, treatment of HeLa cells with curcumin induces the apoptosis (FIG. 5A, following a 24 hours exposure to 75-100 μM of the compound). Since curcumin inhibits p300/CBP HAT activity in vitro, we were interested to find out the effect of curcumin on the acetylation of histones in vivo. Histones were isolated from curcumin-treated cells and subjected to Acetic acid/Urea/Triton (AUT) polyacrylamide gel electrophoresis. Though it is difficult to detect the change of overall acetylation status from the histone fractions, treatment with curcumin modestly increased the unacetylated form of histone H4 and H2B (FIG. 5B, compare lane 2 versus lanes 3 and 4). In order to visualize the effect of curcumin on in vivo histone acetylation more distinctly, histones were hyperacetylated by the treatment with deacetylase inhibitors, TSA and sodium butyrate (FIG. 5B, compare lane 1 versus lane 5). When these cells were treated with 100 μM curcumin, acetylation of histones was found to be inhibited as marked by the appearance of more unacetylated H4 and H2B (FIG. 5B, lane 5 versus 6). In vitro acetylation experiment suggested that p300/CBP mediated acetylation of H3 and H4 was strongly inhibited by curcumin, while the overall analysis of total histones by acetic acid/Urea/Triton polyacrylamide gel electrophoresis did not show a significant change in the histone H3 acetylation. Therefore, we employed western blotting analysis to check the extent of histone acetylation upon curcumin treatment. As depicted in FIG. 5C, acetylation of both H3 and H4 were significantly (6 fold for H3 and 10 fold for H4) (FIG. 5C, lane 5 versus 6) inhibited in the presence of curcumin in vivo. These results convincingly establish curcumin as a potent inhibitor of p300/CBP HAT activity in vitro and in vivo. Since p300/CBP also possesses factor acetyltransferase (FAT) activity and acetylates several nonhistone proteins with functional consequences, we were interested to find out the effect of curcumin on p53 acetylation by p300 in vivo. Cells (293 T) were transfected with p53 and p300 mammalian expression vectors and p53 was immunoprecipitated by anti-p53 monoclonal antibody. Analysis of the immunoprecipitated protein by Western blotting shows that incubation of the cells with curcumin completely inhibits the p300-mediated acetylation of p53 (FIG. 5D, compare lane 2 versus lane 5). Interestingly, p53 could be acetylated by the endogenous FATs even without the transfection of p300 (FIG. 5D, lane 1). However, the endogenous p53 does not seem to get acetylated by the overexpressed p300 (FIG. 5D, lane 3 versus lane 1). This could be due to the fact that both the proteins do not localize together. Significantly, the acetylation status of the endogenous p53 is not altered in the presence of curcumin (FIG. 5D, lane 3 versus lane 6), suggesting that other FATs (GCN5/PCAF/TIP60) (37,38) could acetylate p53 and that this acetylation was not inhibited by curcumin (also see FIG. 5D, compare lane 1 versus lane 4). This result points out to the fact that the in vivo target of curcumin is p300/CBP and not the other FATs.

p53 is often referred to as the ‘guardian of the genome’ and its importance is emphasized by the discovery of mutations of p53 in over 50% of all human cancers. One of the key regulators of p53 function is the acetylation. The acetylation levels of p53 are significantly enhanced in response to every type of stress in vivo (10). This acetylation enhances the activation and stabilization of p53. (39). p53 acetylation is critically important for the recruitment of coactivators (which also includes the acetyltransferases) to promoter regions and the activation of p53-targeted genes in vivo. (40). Therefore, inhibition of p300-specific acetylation of p53 by curcumin should be helpful for the molecular elucidation of acetylation-dependent regulation of p53 function.

Curcumin exhibits a variety of pharmacological effects including anti-tumor, anti-inflamatory and anti-infectious activities. It was found to be a potent inhibitor of the HIV-1 integrase (41). Furthermore curcumin could also inhibit the HIV-1 Tat-mediated transactivation (42) and the UV induced activation of the HIV-LTR gene expression presumably through the inhibition of NFκB activation (43). Though these reports suggest that curcumin may act as a repressor of HIV multiplication, its effect on viral replication was not demonstrated. During the course of infection, HIV genome gets integrated into the human chromatin. A single nucleosome called nuc1 is precisely positioned immediately after the transcription start site in cell lines where HIV promoter is silent. The nuc1 is disrupted during transcriptional activation by acetylation (18). It was elegantly demonstrated that histone deacetylase inhibitors such as trichostatin A, trapoxin, valproic acid and sodium butyrate activate the transcription from HIV promoter. HIV transcriptional activation following the treatment with HDAC inhibitors is associated with nuc1 remodeling (19). Moreover, it has been demonstrated that acetylation of HIV-1 transactivator Tat by p300 is important for its transcriptional activity (22). Thus presumably curcumin would be an effective agent to stop the growth of this virus, through the inhibition of nuc-1 histones and Tat acetylation. We have tested this possibility by investigating the effect of curcumin on syncytia formation upon viral infection to SupT1 cells.

Various experimental formats have been used to evaluate infection of target cells by HIV or inhibition of the viral infection in the presence of an anti-viral compound (44, 45). The standard format is to add titered viral stock to the target cells at a known multiplicity of infection (MOI) in the presence or absence of an anti-viral compound and monitor the synthesis of the viral structural protein p24 or the enzyme reverse transcriptase (46, 47). In the natural context, the viral transfer is more efficient through cell-to-cell contact rather than free virus infecting a target cell. Prevention of the viral transfer between cells is technically more difficult than neutralizing the free virus. SupT1 cells are highly permeable for HIV-1 and these cells make numerous and large syncytia when infected with the virus. Taking advantage of this property, we co-cultured SupT1 cells in the presence of H9/HTLV-IIIb NIH 1983 cells that produce a T-cell tropic virus. A dose-dependent reduction in the number of syncytia was evident with increasing concentration of curcumin and no syncytia were seen at the highest concentration of curcumin (FIG. 6A). To rule out the possibility of cytotoxicity and cell death, we determined the number of viable cells in all the wells using a trypan blue exclusion analysis. The cells were healthy even in the presence of 100 μM curcumin at the end of 48 hrs suggesting that the drug was not cytotoxic for these cells at the concentrations used (data not shown). These results thus show that the p300 specific HAT inhibitor, curcumin inhibits the multiplication of HIV presumably through the inhibition of acetylation of Nuc1 as well as Tat (18, 22). Interestingly, we have found that curcumin strongly inhibits the acetylation of Tat by p300 in vitro (FIG. 6, B and C).

Curcumin is able to inhibit different enzymatic activities that include HIV-1 integrase (41), NFκB activation (43) and p300 specific HAT/FAT activity. Repression of the HIV replication by curcumin could be due to any of the above reasons or a combination thereof. It has been established that histone acetylation (of HIV nuc-1) and the factor (Tat) acetylation is essential for the HIV gene expression as well as multiplication (18, 22, 23). Combinatorial therapeutics has been the only recourse to preventing the spread of HIV, and that too with moderate success. The identification of novel targets, which are involved in the regulation of HIV disease progression, would help in the design of a multipronged therapeutic response aimed at the complete eradication of the virus from the body. In this regard, we have introduced yet another target for the HIV therapeutics the histone acetyltransferase p300/CBP. By regulating the acetylation of Tat and nuc1, this HAT mediates the activation of HIV from its latency. We have also introduced a p300/CBP specific, cell permeable, non-toxic (48) HAT inhibitor, curcumin, which has been shown to inhibit the spread of HIV to the neighboring cells, as highlighted by the syncytia formation assay. These results, in conjunction with earlier studies on HAT inhibition open up a new target with a wide array of potential therapeutic agents in HIV combinatorial therapy. Furthermore, dysfunction of histone acetyltransferases has been found to be associated with several diseases like cardiac hypertrophy, asthma and cancer. In all these diseases it has been found that the cellular histone and nonhistone proteins are hyperacetylated (3-5, 49). Thus, the present finding of curcumin, a non-toxic dietary component, as a p300 specific inhibitor, may find therapeutic applicability for a wide spectrum of diseases, apart from being used as a probe to dissect the molecular pathways in which p300 HAT activity is involved.

### REFERENCES

1. Quivy, V., and Van Lint, C. (2002) Biochem Pharmacol. 64, 925-934.
2. Neely, K. E., and Workman, J. L. (2002) Mol Genet Metab. 76, 1-5.
3. McKinsey, T. A., and Olson, E. N. (2004) Trends Genet. 20, 206-213.
4. Yang, X. J. (2004) Nucleic Acids Res. 32, 959-976.
5. Kramer, 0. H., Gottlicher, M., and Heinzel, T. (2001) Trends Endocrinol Metab. 12, 294-300.
6. Sterner, D. E., and Berger, S. L. (2000) Microbiol. Mol. Biol. Rev. 64, 435-459.
7. Giordano, A., Avantaggiati, M. L. (1999) J Cell Physiol. 181, 218-230.
8. Shikama, N., Lyon, J., and La Thangue, N. B. (1997) Trends Cell Biol. 7, 230-236.
9. Kundu, T. K., Palhan, V. B., Wang, Z., Cole, P. A., and Roeder, R. G. (2000) Mol Cell. 6, 551-561.
10. Brooks, C. L., and Gu, W (2003), Curr Opin Cell Biol. 15, 164-171.
11. Roth, S. Y., Denu, J. M., and Allis, C. D. (2001) Ann. Rev. of Biochem. 70, 81-120.
12. Muraoka, M., Konishi, M., Kikuchi-Yanoshita, R., Tanaka, K., Shitara, N., Chong, J. M., Iwama, T., Miyaki, M. (1996) Oncogene 12, 1565-9.
13. Phillips, A., Vousden, K. H. (2000) Breast Cancer Research 2, 244-246.
14. Murata, T., Kurokawa, R., Krones, A., Tatsumi, K., Ishii, M., Taki, T., Masuno, M., Ohashi, H., Yanagisawa, M., Rosenfeld, M. G., Glass, C. K., and Hayashi, Y. (2001) Hum Mol Genet. 10, 1071-1076
15. Kalkhoven, E., Roelfsema, J. H., Teunissen, H., Den Boer, A., Ariyurek, Y., Zantema, A., Breuning, M. H., Hennekam, R. C., and Peters, D. J. (2003) Hum. Mol. Genet. 12, 441-450.
16. Rouaux, C., Lokic, N., Mbedi Boutiller, S., Leoffler, J. P., and Boutiller, A. L. (2003) EMBO. J 22, 6537-6549
17. Gusterson, R. J., Jazrawi, E., Adcock, I. M., Latchman, D. S. (2003) J. Biol. Chem 278, 6838-47.
18. Van Lint, C., Emiliani, S., Ott, M., and Verdin, E. (1996) EMBO J. 15, 1112-1120.
19. Quivy, V., Adam, E., Collette, Y., Demonte, D., Chariot, A., Vanhulle, C., Berkhout, B., Castellano, R., de Launoit, Y., Burny, A., Piette, J., Bours, V., Van Lint C. (2002) J. Virol. 76, 11091-103.
20. Coull, J. J., He, G., Melander, C., Rucker, V. C., Dervan, P. B., and Margolis, D. M. (2002) J. Virol. 76, 12349-12354.
21. He G and Margolis, D. M. (2002) Mol. Cell. Biol. 22, 2965-2973.
22. Kiernan, R. E., Vanhulle, C., Schiltz, L., Adam, E., Xiao, H., Maudoux, F., Calomme, C., Burny, A., Nakatani, Y., Jeang, K. T., Benkirane, M., and Van Lint, C. (1999) EMBO J. 18, 6106-6118.
23. Ott, M., Schnolzer, M., Garnica, J., Fischle, W., Emiliani, S., Rackwitz, H. R., and Verdin, E. (1999) Curr Biol. 9, 1489-1492.
24. Col, E., Caron, C., Seigneurin-Berny, D., Gracia, J., Favier, A., and Khochbin, S. (2001) J Biol. Chem. 276, 28179-28184.
25. Demonte, D., Quivy, V., Colette, Y., and Van Lint, C. (2004) Biochem Pharmacol. 68, 1231-1238.
26. Lau, O. D., Kundu, T. K., Soccio, R. E., Ait-Si-Ali, S., Khalil, E. M., Vassilev, A., Wolffe, A. P., Nakatani, Y., Roeder, R. G., and Cole P. A. (2000) Mol Cell. 5, 589-595.
27. Cebrat, M., Kim, C. M., Thompson, P. R., Daugherty, M., and Cole, P. A. (2003) Bioorg Med. Chem. 11, 3307-3313.
28. Balasubramanyam, K., Swaminathan, V., Ranganathan, A., and Kundu, T. K. (2003) J Biol. Chem. 278, 19134-19140.
29. Balasubramanyam, K., Altaf, M., Varier, R. A., Swaminathan, V., Ravindran, A., Sadhale, P. P., and Kundu, T. K (2004) J. Biol. Chem. 279, 33716-33726.
30. Kundu, T. K., Wang, Z., and Roeder, R. G. (1999) Mol Cell Biol. 19, 1605-1615.
31. Tachibana, M., Sugimoto, K., Fukushima, T., and Shinkai, Y. (2001) J. Biol. Chem. 276, 25309-25317.
32. Chambers, A. E., Banerjee, S., Chaplin, T., Dunne, J., Debemardi, S., Joel, S. P., and Young, B. D. (2003) Eur J Cancer. 39, 1165-1175.
33. Ryan, C. A., and Annunziato, A. T., (2001) Current Protocols in Molecular Biology (Canada V., ed.) John Wiley and Sons Inc., New York, Chapter 21; Unit 2.2, pp. 2.3-2.10.
34. Bonner, W. M., West, M. H., and Stedman, J. D. (1980) Eur. J. Biochem. 109, 17
35. Lin, J. K., and Lin-Shiau, S. Y. (2001) Proc. Natl. Sci. Counc. ROC (B). 25, 59-66.
36. Pan, M. H., Chang, W. L., Lin-Shiau, S. Y., Ho, C. T., and Lin, J. K. (2001) J Agric Food Chem. 49, 1464-1474.
37. Ard, P. G., Chatterjee, C., Kunjibettu, S., Adside, L. R., Gralinski, L. E., and McMahon S B. (2002) Mol Cell Biol. 22, 5650-5661.
38. Wang, T., Kobayashi, T., Takimoto, R., Denes, A. E., Snyder, E. L., el-Deiry, W. S., and Brachmann, R. K. (2001), EMBO J. 20, 6404-6413.
39. Ito, A., Lai, C. H., Zhao, X., Saito, S., Hamilton, M. H., Appella, E., and Yao, T. P. (2001), EMBO J. 20, 1331-1340.
40. Barlev, N. A, Liu, L., Chehab, N. H., Mansfield, K., Harris, K. G., Halazonetis, T. D., Berger, S. L. (2001), Mol Cell, 8, 1243-1254.
41. Mazumder, A., Raghavan, K., Weinstein, J., Kohn, K. W., Pommier, Y. (1995) Biochem Pharmacol. 49, 1165-1170.
42. Barthelemy, S., Vergnes, L., Moynier, M., Guyot, D., Labidalle, S., and Bahraoui E. (1998) Res Virol. 149, 43-52.
43. Taher M M, Lammering G, Hershey C, Valerie K. (2003) Mol Cell Biochem, 254, 289-297.
44. McKeating, J. A., McKnight, A., McIntosh, K., Clapham, P. R., Mulder, C., and Weiss, R. A. (1989) J. Gen. Virol. 70, 3327-3333.
45. Gera, A., Spiegel, S., and Loebenstein, G. (1986) Methods Enzymol., 119, 729-734.
46. Kanamoto, T., Kashiwada, Y., Kanbara, K., Gotoh, K., Yoshimori, M., Goto, T., Sano, K., and Nakashima, H. (2001) Antimicrob. Agents Chemother., 45, 1225-1230.
47. Japour, A. J., Fiscus, S. A., Arduino, J. M., Mayers, D. L., Reichelderfer, P. S., and Kuritzkes, D. R. (1994) J. Clin. Microbiol. 32, 2291-2294.
48. Cheng, A. L, Hsu, C. H., Lin, J. K., Hsu, M. M., Ho, Y. F., Shen, T. S., Ko, J. Y., Lin, J. T., Lin, B. R., Ming-Shiang, W., Yu, H. S., Jee, S. H., Chen, G. S., Chen, T. M., Chen, C. A., Lai, M. K., Pu, Y. S., Pan, M. H., Wang, Y. J., Tsai, C. C., and Hsieh, C. Y. (2001) Anticancer Res. 21, 2895-2900.
49. Barnes, P. J., Ito, K., and Adcock, I. M. (2004), Lancet. 363, 731-733.

### FIGURE LEGENDS

FIG. 1

Curcumin is a Potent Inhibitor of Histone Acetyltransferase p300

HAT assays were performed either with p300, CBP or PCAF in the presence or absence of curcumin using core histones (800 ng) and processed for filter binding (A) or fluorography (B, C and D). (B, C & D), core histones without any HAT (Lane 1), histones with HAT (lane 2), with HAT and in presence of DMSO as solvent control (lane 3), HAT in presence of 20, 40, 60, 80 μM concentrations of curcumin respectively (lanes 4-7).

FIG. 2

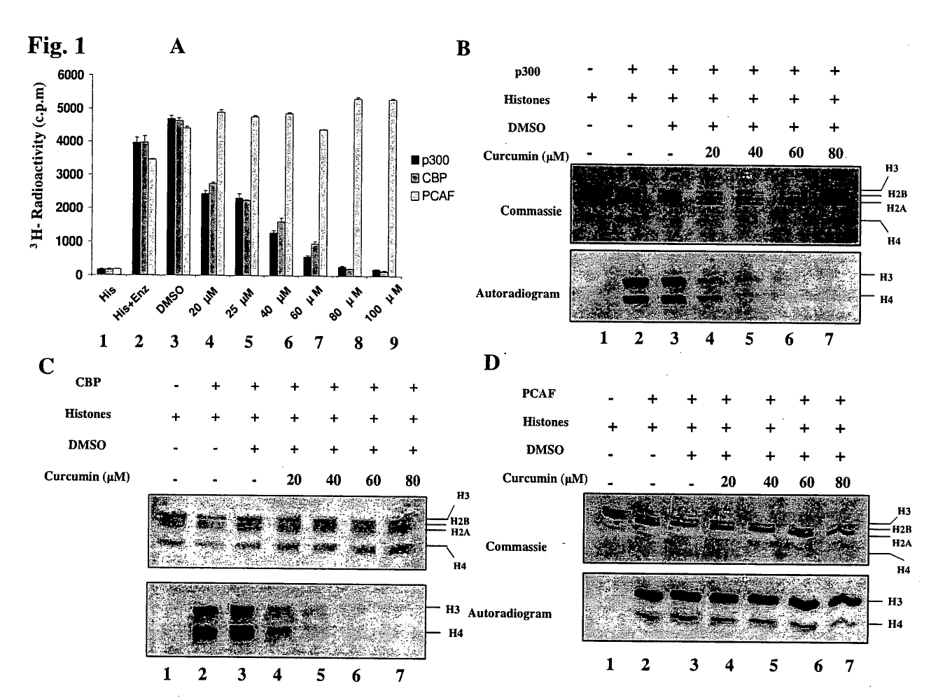

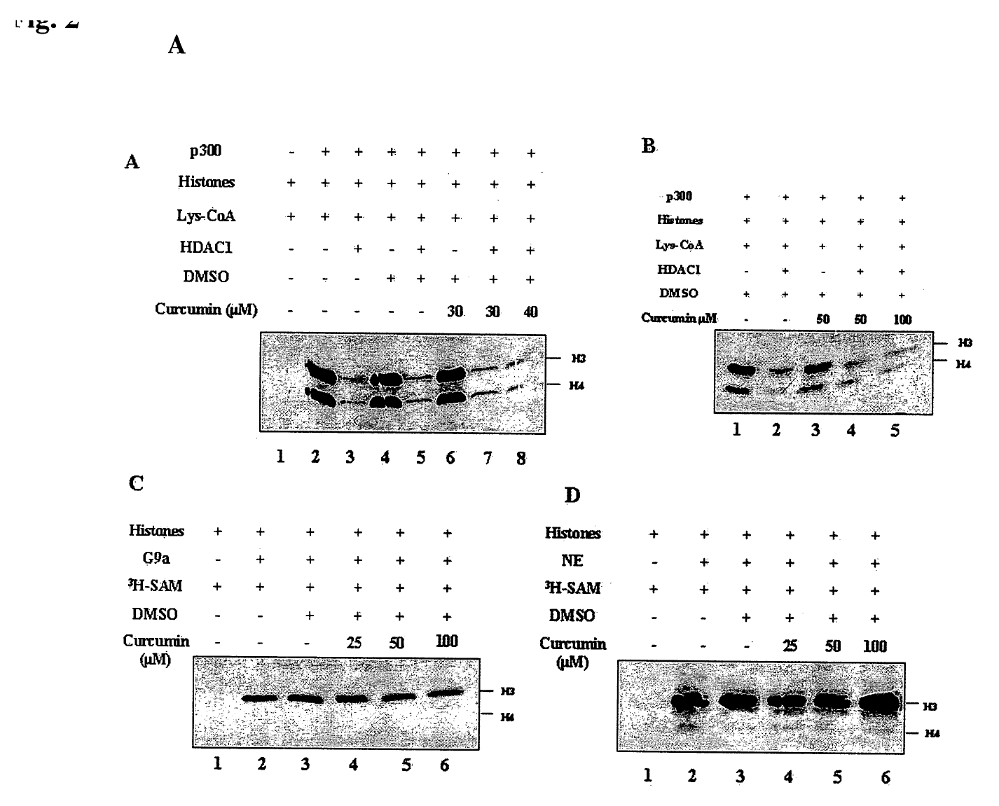

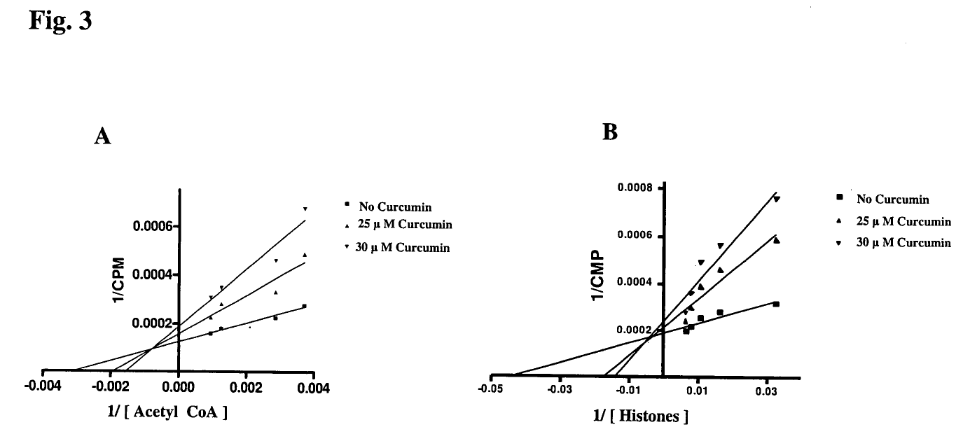

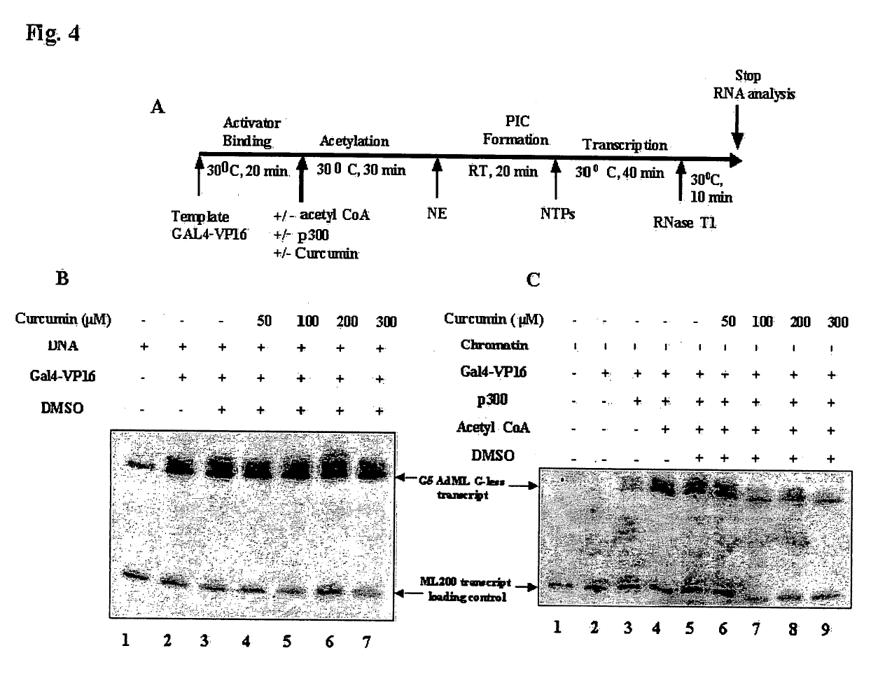

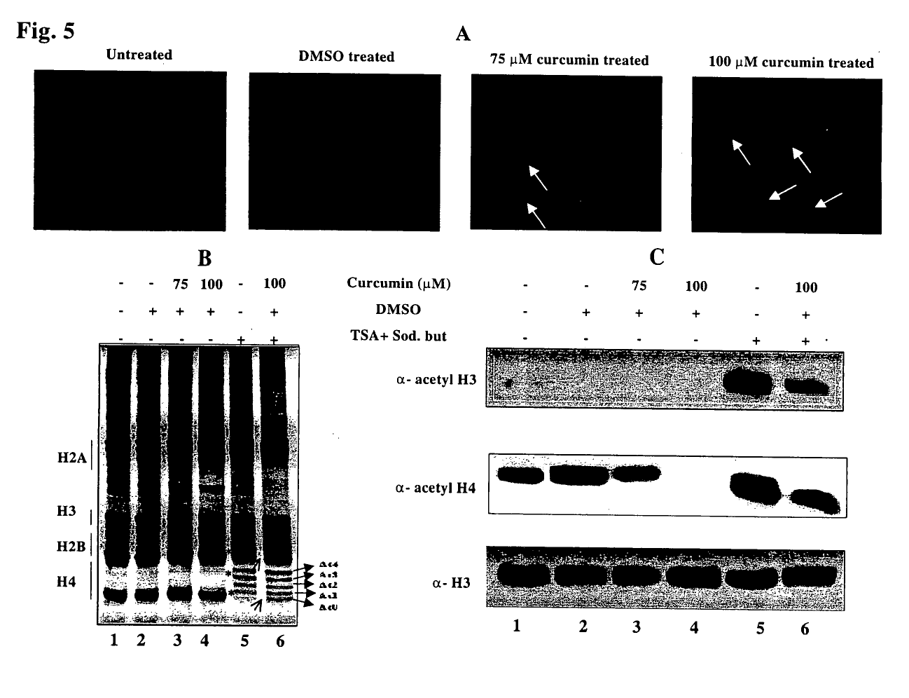

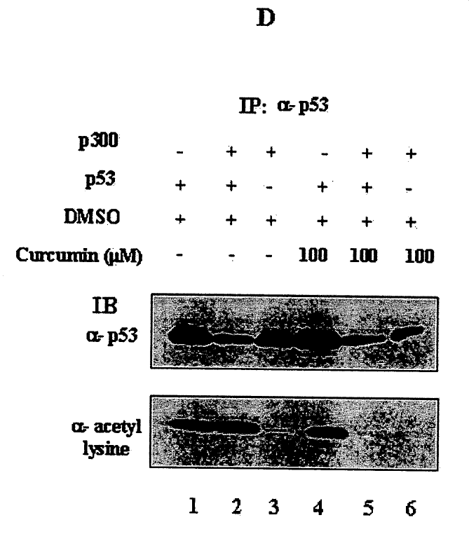

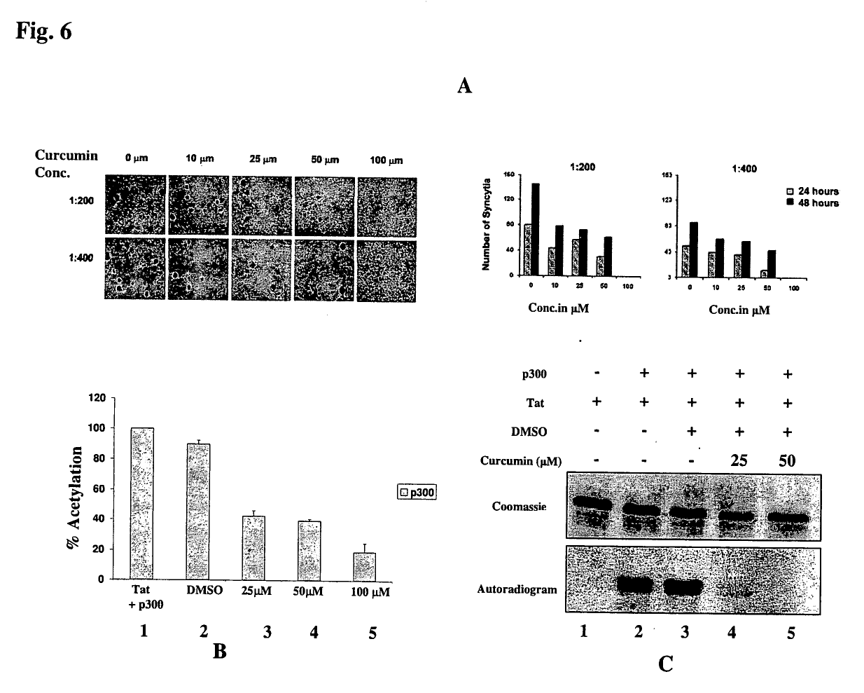

Histone deacetylase (A) and methyltransferase (B and C) activities are not affected by curcumin. (A) Acetylated (by p300) HeLa core histones were subjected to deacetylation with 60 ng of recombinant HDAC 1 in the presence (30 and 40 μM) or absence of curcumin. Lane 1, unlabelled histones; lane 2, ³H-labelled acetylated histones; lane 3, acetylated histones treated with HDAC1; lane 4, acetylated histones treated with DMSO; lane 5, deacetylation of histones in presence of DMSO; lane 6, acetylated histones with curcumin (30 μM); lanes 7 and 8, deacetylation of acetylated histones by HDAC1 in presence of 30 μM and 40 μM curcumin respectively. (B) Acetylated (by p300) HeLa core histones were subjected to deacetylation with 60 ng of recombinant HDAC 1 in the presence (50 and 100 μM) or absence of curcumin. Lane 1, acetylated histones treated with DMSO; lane 2, deacetylation of histones in presence of DMSO; lane 3, acetylated histones with curcumin (50 μM); lanes 4 and 5, deacetylation of acetylated histones by HDAC1 in presence of 50 μM and 100 μM curcumin respectively. Histone Methyltransferase (HMTase) assays were performed in 30 μl reaction in the presence or absence of curcumin using either G9a (B) or (C) NE as the enzyme sources. The reaction products were TCA precipitated, resolved on 15% SDS-PAGE and subjected to fluorography followed by autoradiography. (B and C) Lane 1, histones; lane 2, histones with enzyme (NE/G9a), lane 3: histones with the enzymes and DMSO, lanes 4-6: histones with the enzymes and in the presence of 25, 50 and 100 μM concentrations of curcumin respectively.

FIG. 3.

Inhibition Kinetics of Curcumin for p300 (A) Line weaver—Burk plot (LB) showing the effect of curcumin on p300 mediated acetylation of core histones. HAT assays were carried out with a fixed concentration, of [³H]-acetyl CoA (354 nM) and increasing concentrations of histones (0.033-0.165 μM) in the presence (25 and 30 μM) or absence of curcumin. (B) depicting the same LB plot representation of curcumin effect on p300 HAT activity at a fixed concentration of histone (8 pm) and increasing concentration of [³H]-acetyl CoA in the presence (25 and 30 μM) or absence of curcumin. The results were plotted using the Graph Pad Prism software.

FIG. 4.

Curcumin Inhibits p300 Hat Activity Dependent Transcriptional Activation from Chromatin Template.

(A), Schematic representation of the in vitro transcription protocol. In vitro transcription from naked DNA (B) and chromatin template (C). 30 ng DNA and freshly assembled chromatin template (equivalent to 30 ng of DNA) were subjected to the protocol in panel A with or without curcumin, 50 ng of Gal4-VP16, 25 ng of baculovirus-expressed highly purified His₆-tagged p300 (full length) and 1.5 μM acetyl CoA. The in vitro transcription reaction mixtures were analyzed on 5% urea-acrylamide gel and further processed by autoradiography. (B) Lane 1, without activator (basal transcription); lane 2, with activator (Gal4-VP16); lane 3, with activator and DMSO; lanes 4-7, with activator and 50, 100, 200, 300 μM curcumin respectively. (C) Lane 1, without activator; lane 2, with activator; lane 3, with activator and p300, lane 4, with activator, p300 and acetyl CoA, lane 5, reaction of lane 4 in presence of DMSO, lanes 6-9, reaction of lane 4 in presence 50, 100, 200, 300 μM concentration of curcumin.

FIG. 5.

Curcumin Induces Apoptosis (a) and Inhibits Acetylation of Histones and P53 In Vivo (B, C and D).

(A) Hoechst staining of untreated HeLa cells and cells treated with DMSO and 75 and 100 μM curcumin. Arrows indicate apoptotic nuclear fragmentation

(B) HeLa cells were treated as indicated, for 24 hours, histones were extracted and separated on an 18% acid/urea/triton (AUT) PAGE. The protein bands were visualized by Coomassie brilliant blue staining. Lane 1, histones extracted from untreated cells, lane 2, DMSO (solvent control) treated cells, lane 3 curcumin (75 μM) treated cells, lane 4, curcumin (100 μM) treated cells, lane 5, trichostatin (2 μM) and sodium butyrate (10 mM) treated cells and lane 6, trichostatin A (2 μM), sodium butyrate (10 mM) and curcumin (100 μM) treated cells are shown. Asterisk (*) indicates hyperacetylation of histones in response to HDAC inhibition by TSA and sodium butyrate. Arrow (→) indicates inhibition of TSA-induced hyperacetylation of H4 and H2B by curcumin. (C). The acid-extracted histones were resolved over 12% SDS-PAGE and were analyzed by western blot using antibodies against acetylated histone H3 and H4. Loading and transfer of equal amounts of protein were confirmed by immunodetection of histone H3. (D) 293 T cells were transiently transfected with CMV-p53 and CMV-p300 either alone or in combination as indicated. The transfected cells were then treated with curcumin (100 μM) or vehicle (DMSO) for 24 hours. The p53 protein was immunoprecipitated from the cell lysates using p53 monoclonal antibody and acetylation status was analyzed by western blotting. Lanes 1 and 4, transfection with p53 alone; lanes 2 and 5, co-transfection with p53 and p300; lanes 3 and 6, transfection with p300 alone. (IP:Immunoprecipitation and IB: Immunoblotting).

FIG. 6.

Repression of HIV Multiplication Through Inhibition of Syncytium Formation in the Presence of Curcumin.

(A), H9/HTLV-IIIb NIH 1983 carrying stable integrated virus were cocultured with excess numbers of SupT1 cells at 1:200 or 1:400 ratio. A total of 0.1×10⁶ cells were seeded per well. Curcumin in DMSO was added to the wells to the final concentration as shown and the cultures were incubated at 37° C. Formation of syncytia is visible under light microscope within 12 hrs. The total number of syncytia in 10 representative wells was counted and presented. The data are representative of 2 independent experiments.

(B), (C) Curcumin inhibits acetylation of Tat protein by p300. HAT assays were performed with p300 in the presence or absence of curcumin using purified Tat protein (2 μg) and processed for filter binding (B) or fluorography (C) as mentioned earlier except that the reaction mixture was incubated at 30° C. for 40 minutes. (B), the percentage of acetylation was calculated in each of the cases by accounting for the corrections of acetylation in the case of Tat alone. (C), Lane 1, Tat alone; lane 2, Tat with p300; lane 3, Tat with p300 and in presence of DMSO (as solvent control); lanes 4 and 5, Tat with p300 and in presence of 25 and 50 μM of curcumin respectively.

Isolation and Purification of Curcumin from Curcuma Longa:

Twenty grams of Curcuma longa in 100 ml of dichloromethane was mechanically stirred and refluxed for one hour. The mixture was suction filtered and the filtrate was concentrated in rotary evaporator maintained at 50° C. The reddish yellow oily residue was trituted with 20 mL of hexane and the resulting solid was collected by suction filtration. The crude material obtained after tritution with hexane was dissolved in minimum amount of 99% dichloromethane-1% methanol (v/v) and loaded onto a column packed with 75 gm of silica gel. The column was eluted with the same solvent. The fractions containing least polar colored components were combined and solvents were removed on a water bath to give curcumin (mp 178-182° C.), ¹H NMR (DMSO-d₆) d 3.90 (6H, s, OCH₃), 6.06 (1H, s, C(OH)=CH), 6.76 (2H, d 2.6), 7.32 (2H, s), 2H, d, 17-H), 9.70 (2 h, Phenolic OH)

Preparation of Curcumin Derivatives:

To solution of curcumin in acetone was treated with hallo compounds for 10-20 hours, in the presence of K₂CO₃. If the reactions were not completed reflux the reaction mixtures for few hours, and then the solvent was removed in vacuo. The products were purified by column chromatography and products were characterized by NMR spectroscopy.

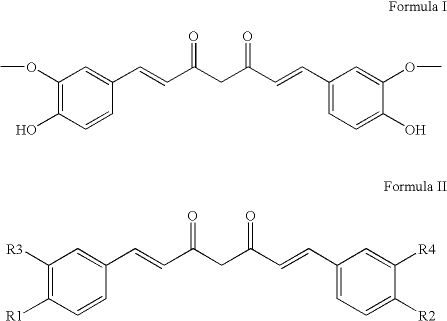

Compounds of structural formula m as specific inhibitors of histone acetyl transferase where in
R₁ is Hydroxy, O-Methoxy, O-Ethoxy, O—CH₂—COOH, O—CO—CH₂—Cl, O—SO₂—CH₃, O—CO—CH₃,
R₂ is Hydroxy, O-Methoxy, O-Ethoxy, O—CH₂—COOH, O—CO—CH₂—Cl, O—SO₂—CH₃, O—CO—CH₃,
R₃ O-Methoxy
R₄ O-Methoxy
R₅ is CO, ═N—OH
R₆ is CO, ═N—OH

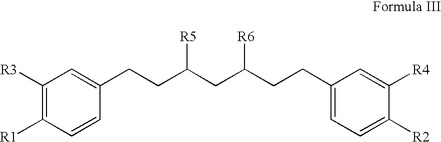

Compounds of structural formula II as specific inhibitors of histone acetyl transferase where in
R₁ is O-Methoxy, O-Ethoxy, O-Isopropoxy, O-Allyoxy, O-Butoxy, O-t-Butoxy, O—CH₂—COOH, O—CO—CH₂—Cl, O—SO₂—CH₃, O—CO—CH₃, O—CO—CH₂—CH₃, ONa, OH, O—CH₂OH, OK
R₂ is O-Methoxy, O-Ethoxy, O-Isopropoxy, O-Allyoxy, O-Butoxy, O-t-Butoxy, O—CH₂—COOH, O—CO—CH₂—Cl, O—SO₂—CH₃, O—CO—CH₃, O—CO—CH₂—CH₃, ONa, OH, O—CH₂OH, OK
R₃ O-Methoxy, OH, O-Ethoxy, O-Isopropoxy, O—CO—CH₃, O—SO₂—CH₃, O—CH₂—COOH, O—CH₂OH, OK
R₄ O-Methoxy, OH, O-Ethoxy, O-Isopropoxy, O—CO—CH₃, O—SO₂—CH₃, O—CH₂—COOH, O—CH₂OH, OK

1. Use of 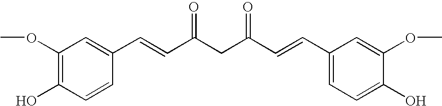
as a histone acetyltransferase inhibitor.

2. Use of 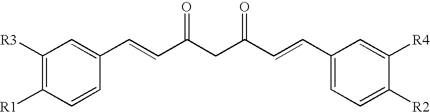
as a histone acetyltransferase inhibitor, wherein
R₁ is one of O-Methoxy, O-Ethoxy, O-Isopropoxy, O-Allyoxy, O-Butoxy, O-t-Butoxy, O—CH₂—COOH, O—CO—CH₂—Cl, O—SO₂—CH₃, O—CO—CH₃, O—CO—CH₂—CH₃, ONa, OH, O-Ethoxy, O-Isopropoxy, O—CO—CH₃, O—SO₂—CH₃, O—CH₂—COOH, O—CH₂OH, OK;
R₂ is one of O-Methoxy, O-Ethoxy, O-Isopropoxy, O-Allyoxy, O-Butoxy, O-t-Butoxy, O—CH₂—COOH, O—CO—CH₂—Cl, O—SO₂—CH₃, O—CO—CH₃, O—CO—CH₂—CH₃, ONa, OH, O-Ethoxy, O-Isopropoxy, O—CO—CH₃, O—SO₂—CH₃, O—CH₂—COOH, O—CH₂OH, OK;
R₃ is one of O-Methoxy, OH, O-Ethoxy, O-Isopropoxy, O—CO—CH₃, O—SO₂—CH₃, O—CH₂—COOH, O—CH₂OH, OK; and
R₄ is one of O-Methoxy, OH, O-Ethoxy, O-Isopropoxy, O—CO—CH₃, O—SO₂—CH₃, O-CH₂-COOH, O-CH₂OH, OK.

3. Use of 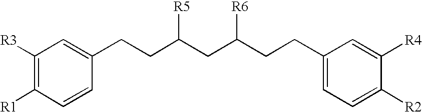
as a histone acetyltransferase inhibitor, wherein
R₁ is one of Hydroxy, O-Methoxy, O-Ethoxy, O—CH₂—COOH, O—CO—CH₂—Cl, O—SO₂—CH₃, O—CO—CH₃,
R₂ is one of Hydroxy, O-Methoxy, O-Ethoxy, O—CH₂—COOH, O—CO—CH₂—Cl, O—SO₂—CH₃, O—CO—CH₃,
R₃ is O-Methoxy
R₄ is O-Methoxy
R₅ is one of CO, ═N—OH
R₆ is one of CO, ═N—OH.
